(19) United States
(12) Patent Application Publication
(10) Pub. No.: US 2004/0204631 A1
(43) Pub. Date: Oct. 14, 2004

(54) Tongue depressor

(75) Inventor: Kiyoshi Yasuda, Ichihara-shi (JP)

Correspondence Address: JORDAN AND HAMBURG LLP, 122 EAST 42ND STREET, SUITE 4000, NEW YORK, NY, 10168, US

(73) Assignees: Fine Co., Ltd., Shinagawa-ku (JP); Kiyoshi Yasuda, Ichihara-shi (JP)

(21) Appl. No.: 10/689,353

(22) Filed: Oct. 20, 2003

(30) Foreign Application Priority Data
Apr. 9, 2003 (JP) 2003-137970

(51) Int. Cl. A61B 1/267

(52) U.S. Cl. 600/199

(57) ABSTRACT

There is disclosed a tongue depressor which has a light function to visually examine or clean the inside of a mouth easily and effectively, and which can be operated even by one hand, and light deep teeth or a throat part without any reductions in the amount of a light. Because of a structure in which a hole is bored in the main body of the tongue depressor, and a lighting fixture such as a commercially available penlight can be inserted/pulled out, two functions, i.e., a tongue depressor and lighting, can be provided. A shape is simple, fabrication costs are low, and sanitary supervision is easy. Since the tongue depressor can be operated by one hand, mouth cleaning can be carried out by the other hand.

### BACKGROUND OF THE INVENTION

1. Field of the Invention

The present invention relates to a tongue depressor which lights the inside of a mouth to press a tongue or the like when the tongue, teeth or a throat in the mouth is visually examined or cleaned.

2. Description of the Related Art

As it is dark in the mouth, the inside of the mount must be lit in the case of affection in the mouth or dental visual examination.

Conventionally, a reflecting mirror has been mounted on a head, and a light has been converged thereon to be reflected, whereby the inside of the mouth has been lit. On the other hand, in order to examine a throat part or a deep tongue part, the tongue must be pushed down by a thin spatula-shaped tongue depressor.

When the teeth or the outside of a gum is visually examined, cheeks must be pressed from the inside to the outside by the tongue depressor, or lips must be everted. Lighting is necessary in such a case, but the lighting and the tongue depressor have conventionally been separate.

Accordingly, a tongue depressor which has a lighting function has been invented. For example, it is described in Japanese Utility Model Publication No. 28963/1987 and Japanese Utility Model Application Laid-open No. 68719/1994. However, such a tongue depressor is constituted to be inserted into/pulled out from a holder which comprises a power source and a light source. Consequently, fabrication costs are high for the holder and the tongue depressor, and shapes thereof are complex. Furthermore, the tip of the tongue depressor and the light source in the holder must be set structurally part from each other. Thus, there are problems such as a reduction in the amount of a light before the light reaches the tip of the tongue depressor.

### SUMMARY OF THE INVENTION

The present invention provides a tongue depressor which has light function to enable lighting of deep teeth or a throat part without any reductions in the amount of a light when the inside of a mount is visually examined or cleaned. The shape and the operation of this tongue depressor are simple. The tongue depressor can be operated by one hand, while cleaning can be carried out by the other hand.

The tongue depressor can be fabricated inexpensively, its sanitary supervision is easy after use, and it can be conveniently used even by a helper.

Since a lighting fixture such as a commercially available penlight can be inserted into the main body of the tongue depressor, no holder is necessary, and the functions of lighting and a tongue depressor can be provided.

### BRIEF DESCRIPTION OF THE DRAWINGS

FIG. 1 is a perspective view of a state in which a lighting fixture 7 is fixed in a main body 1.

FIG. 2 is a side view of the main body 1, in which 2 is a top surface, 3 is a bottom surface, 8 is a hole, 9 is a slope, and 10 is a convex part.

FIG. 3 is a plan view of the main body 1, in which 4 is a side face, 5 is a convergent part, and 11 is a flange.

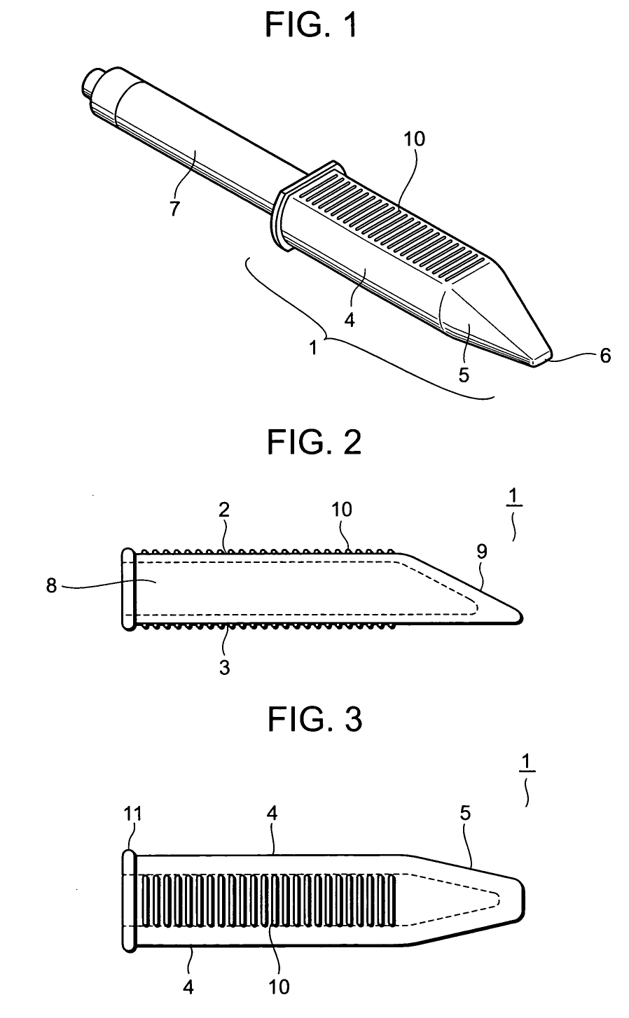

### DESCRIPTION OF THE EMBODIMENT

Next, the embodiment of the present invention will be described with reference to the accompanying drawings.

The main body 1 of a tongue depressor is made of a high heat-resistant material which has light transmissivity, such as a polyethylene terephthalate resin. The main body 1 is set to a length to be inserted into the deep part of a mouth. A width of each of the side faces 4 of both sides is set larger than a thickness (bulk) between the top surface 2 and the bottom surface 3 of the main body 1. The main body 1 is formed to be narrower in width from the vicinity of the convergent part 5 to a tip 6.

The side face 4 is formed in a semicircular shape swollen to the outside. The hole 8 is bored in the main body 1 to allow insertion/pulling-out of a lighting fixture 7. The slope 9 is disposed from the vicinity of the convergent part 5 on the top surface of the main body 1. It is brought into contact with the bottom surface to close the hole 8. The tip 6 is disposed on the front side. A commercially available penlight or the like which has a power source and a light source is used for the lighting fixture 7. A plurality of line convex parts 10 are disposed on the top surface 2 and the bottom surface 3 excluding the convergent part 5 and the side faces 4. The flange 11 is disposed on the rear end of the main body 1.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description and all changes which come within the meaning and range of equivalency of the claims are therefore intended to be embraced therein.

### Method of Use

According to a method of use, the lighting fixture 7 is inserted into the hole 8. The lighting fixture 7 is turned ON. The tip 6 is first inserted when the light fixture 7 is inserted between teeth. A light of the lighting fixture 7 is transmitted by the tip 6 and the convergent part 5 to light the inside of the mouth. To light a throat part or a deep tongue part, the main body 1 is inserted more deeply to press the tongue by the tip 6. Each of the convex parts 10 is a slip stopper when a patient bites.

To light deep teeth or the outer surface of a gum, the main body 1 is inserted into the inside of cheeks or lips to press them from the inside to the outside. An opening width becomes narrow if there are no upper and lower teeth. In such a case, the main body 1 is rotated by 90°, and the side faces 4 of both sides are bitten to expand the opening width.

The conventional tongue depressor which has the lighting function is classified into the holder and the tongue pressing plate, which are connected to each other. The tongue depressor of the invention is different in constitution from the conventional tongue depressor since the lighting fixture is inserted into the main body. As a result, the tongue depressor of the invention can be inexpensively fabricated by a structure much simpler than that of the conventional case. If the hole is enlarged, many commercially available penlights or the like can be inserted/pulled out. Furthermore, since the light source is located near the tip of the tongue depressor, almost no reductions occur in the amount of a light, and a lighting range is wide. The opening width can be expanded by rotating the main body.

Sanitary supervision is easy because of a simple form. Even a helper who is a member of a family can use the tongue depressor. The use of the high heat-resistant material enables inexpensive sterilization, and thus it can be disposable.

What is claimed is:

1. A tongue depressor which has a lighting function, comprising:
a hole bored in a longitudinal direction of a main body.

2. The tongue depressor according to claim 1, wherein the hole bored in the main body is closed on a front part of the main body to make the main body light-transmissive.

3. A tongue depressor comprising:
a hole bored in a longitudinal direction of a main body which uses a light-transmissive material,
wherein a light fixture can be inserted into/pulled out from the main body.
